(19) United States
(12) Patent Application Publication
(10) Pub. No.: US 2024/0336603 A1
(43) Pub. Date: Oct. 10, 2024

(54) PROCESS FOR THE PREPARATION OF A CYP11A1 INHIBITOR AND INTERMEDIATES THEREOF

(71) Applicant: ORION CORPORATION, Espoo (FI)

(72) Inventors: Oskari Karjalainen, Espoo (FI); Miika Karjomaa, Espoo (FI)

(21) Appl. No.: 18/573,360

(22) PCT Filed: Jun. 22, 2022
(86) PCT No.: PCT/FI2022/050446

§ 371 Date: Dec. 21, 2023

(30) Foreign Application Priority Data
Jun. 23, 2021 (FI) 20215736

(51) Int. Cl. C07D 405/14

(52) U.S. Cl. CPC C07D 405/14

(57) ABSTRACT

The present invention relates to an improved process for the preparation of 4H-pyranone structured CYP11A1 inhibitors such as 5-((1-(methylsulfonyl)-piperidin-4-yl)methoxy)-2-((5-(trifluoromethyl)isoindolin-2-yl)methyl)-4H-pyran-4-one (1A) and key intermediates thereof including 5-(trifluoromethyl)isoindoline (V) and 5-hydroxy-2-((5-(trifluoromethyl)isoindolin-2-yl)methyl)-4H-pyran-4-one (III). CYP11A1 inhibitors are useful in the treatment of hormonally regulated cancers, such as prostate cancer and breast cancer.

### TECHNICAL FIELD

The present invention relates to an improved process for the preparation of 4H-pyranone structured CYP11A1 inhibitors such as 5-((1-(methylsulfonyl)-piperidin-4-yl)methoxy)-2-((5-(trifluoromethyl)isoindolin-2-yl)methyl)-4H-pyran-4-one (1A) and key intermediates thereof including 5-(trifluoromethyl)isoindoline (V) and 5-hydroxy-2-((5-(trifluoromethyl)isoindolin-2-yl)methyl)-4H-pyran-4-one (III).

### BACKGROUND OF THE INVENTION

The compound 5-((1-(methylsulfonyl)piperidin-4-yl)methoxy)-2-((5-(tri-fluoromethyl)isoindolin-2-yl)methyl)-4H-pyran-4-one of formula (1A) and derivatives thereof have been disclosed in WO 2018/115591. Compound of formula (1A) is a selective inhibitor of CYP11A1 enzyme and is useful in the treatment of hormonally regulated cancers, such as prostate cancer and breast cancer.

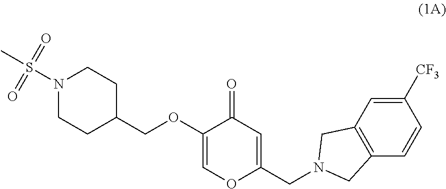 [SMILES: CS(=O)(=O)N1CCC(COc2coc(CN3Cc4ccc(C(F)(F)F)cc4C3)cc2=O)CC1]

WO 2018/115591 discloses a process for the preparation of the compound of formula (1A) according to Scheme 1.

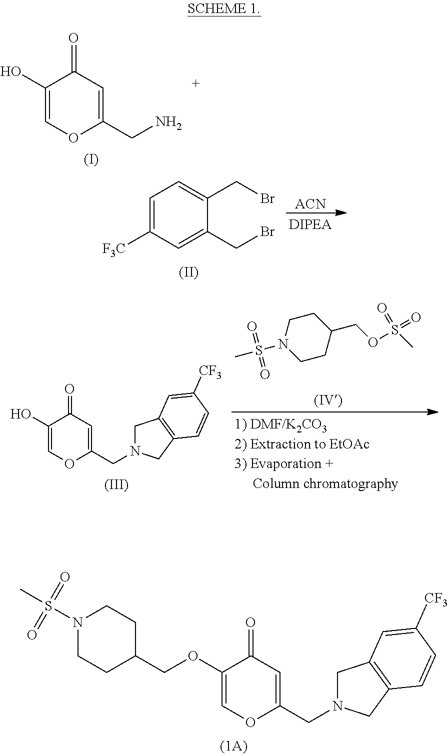

This process comprises reacting 2-(aminomethyl)-5-hydroxy-4H-pyran-4-one (I) with 1,2-bis(bromomethyl)-4-(trifluoromethyl)benzene (II) in acetonitrile in the presence of N,N-diisopropylethylamine (DIPEA) to obtain 5-hydroxy-2-((5-(tri-fluoromethyl)isoindolin-2-yl)methyl)-4H-pyran-4-one (III) followed by reaction with (1-(methylsulfonyl)piperidin-4-yl)methyl methane sulfonate (IV′) in dimethyl-formamide (DMF) in the presence of potassium carbonate. The compound of formula (1A) is recovered from the reaction mixture by addition of water, extracting with ethyl acetate and evaporating to dryness followed by purification by column chromatography.

The above mentioned process has several drawbacks. The compound of formula (II) is not commercially available and is a strong lachrymator and therefore difficult to handle. The compound of formula (I) is also not commercially available and its manufacture has proved to be difficult. It has been manufactured by azidation of 2-(chloromethyl)-5-hydroxy-4H-pyran-4-one followed by treatment with HBr in a mixture of acetic acid and phenol (Atkinson, J. et al, Journal of Medicinal Chemistry, 1979, 22, 1, 99-106). However, the formed azide intermediate is susceptible to explosion and therefore the process is not suitable for use in the large scale. An alternative route, amination of 5-(benzyloxy)-2-[(p-toluenesulfonyloxy)methyl]-4H-pyran-4-one followed by debenzylation yields compound of formula (I) but in low yield (Atkinson, J. et al, Journal of Medicinal Chemistry, 1979, 22, 1, 99-106).

The final step to produce compound of formula (1A) suffers from the need to evaporate the solvent to dryness for obtaining the brownish crude product, which needs to be purified by column chromatography leading to poor yield. The process does not provide the possibility of crystallization the end product directly from the reaction mixture.

Methods for preparing 5-(trifluoromethyl)isoindoline (V) have been disclosed earlier in U.S. Pat. Nos. 7,345,180, 8,299,021, Van Goethem, S. et al., Bioorg. Med. Chem. Lett., 18 (2008), 4159-4162 and Pinard, E. et al, Bioorg. Med. Chem. Lett., 20 (2010), 6960-6965. These methods suffer from low yields and lack of commercially available starting materials.

Thus, there is a need for a more practical and economical process that is suitable for the manufacture of compound (1A) or a pharmaceutically acceptable salt thereof as well as intermediates thereof in the large scale.

### SUMMARY OF THE INVENTION

It has now been found that the compound of formula (1A) or a pharmaceutically acceptable salt thereof can be prepared using a process and a synthesis route, which is more practical, economical and suitable for use in a large scale. Intermediate compounds are prepared with improved methods utilizing commercially available starting materials and resulting in acceptable yields. Moreover, the compound of formula (1A) is obtained as a high purity and low colored product directly by crystallization without the need of purification by chromatography.

Thus, in one aspect, the present invention provides a process for the preparation of a compound of formula (V) or a pharmaceutically acceptable salt thereof

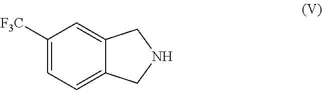 [SMILES: FC(F)(F)c1ccc2c(c1)CNC2]

comprising the steps of either
   a) treating a compound of formula (VI)

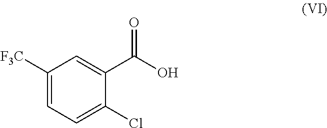 [SMILES: O=C(O)c1cc(C(F)(F)F)ccc1Cl]

with copper cyanide to obtain a compound of formula (VII)

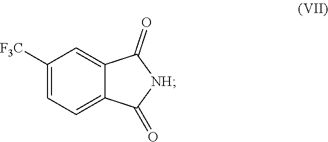 [SMILES: O=C1NC(=O)c2cc(C(F)(F)F)ccc21]

b) protecting the compound of formula (VII) to obtain a compound of formula (VIII) wherein Pg is a protecting group

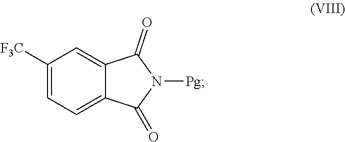

c) reducing the compound of formula (VIII) to obtain a compound of formula (IX)

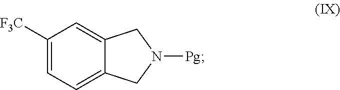

d) deprotecting the compound of formula (IX) to obtain a compound of formula (V); and optionally converting it to its pharmaceutically acceptable salt;
or
a′) treating a compound of formula (VI)

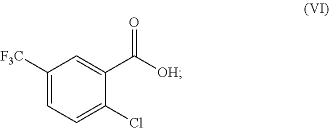 [SMILES: O=C(O)c1cc(C(F)(F)F)ccc1Cl]

with hexacyanoferrate[II] salt in the presence of a catalyst to obtain a compound of formula (X) or a pharmaceutically acceptable salt thereof

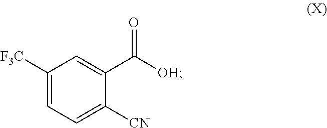 [SMILES: N#Cc1ccc(C(F)(F)F)cc1C(=O)O]

b′) reducing the compound of formula (X) or a pharmaceutically acceptable salt thereof to obtain a compound of formula (XI) or a pharmaceutically acceptable salt thereof

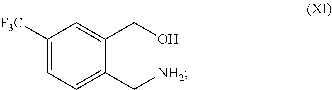 [SMILES: NCc1ccc(C(F)(F)F)cc1CO]

c′) treating the compound of formula (XI) or a pharmaceutically acceptable salt thereof with thionyl chloride to obtain a compound of formula (V); and optionally converting it to its pharmaceutically acceptable salt.

In another aspect, the present invention provides a process for the preparation of a compound of formula (1A) or a pharmaceutically acceptable salt thereof

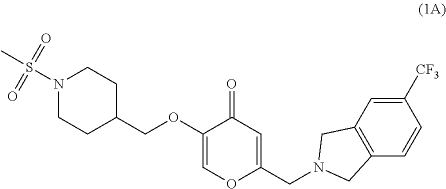 [SMILES: CS(=O)(=O)N1CCC(COc2coc(CN3Cc4ccc(C(F)(F)F)cc4C3)cc2=O)CC1]

comprising the steps of
   i) preparing hydrochloride salt of 5-(trifluoromethyl)isoindoline (V) according to any of the processes as defined above;
   ii) reacting hydrochloride salt of 5-(trifluoromethyl)isoindoline (V) with a compound of formula (XII)

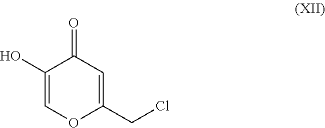

to produce a compound of formula (III);

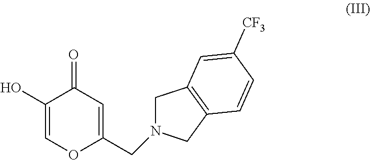 [SMILES: O=c1cc(CN2Cc3ccc(C(F)(F)F)cc3C2)occ1O]

iii) reacting a compound of formula (III) with a compound of formula (IV)

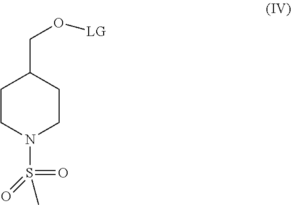

wherein LG is a leaving group selected from a mesyl or a tosyl group,
to produce compound of formula (1A), and optionally converting it to its pharmaceutically acceptable salt.

In still another aspect, the present invention provides a process for the preparation of a compound of formula (III)

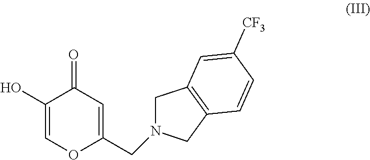 [SMILES: O=c1cc(CN2Cc3ccc(C(F)(F)F)cc3C2)occ1O]

comprising the steps of
   a″) reacting a compound of formula (XII)

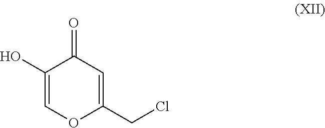

with hydrochloride salt of 5-(trifluoromethyl)isoindoline (V) in dimethyl sulfoxide in the presence of N,N-diisopropylethylamine;
   b″) adding isopropyl alcohol and water to the mixture; and
   c″) isolating the compound of formula (III).

In still another aspect, the present invention provides a process for the preparation of a compound of formula (1A) or a pharmaceutically acceptable salt thereof

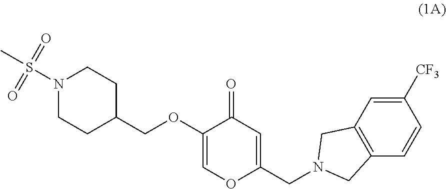 [SMILES: CS(=O)(=O)N1CCC(COc2coc(CN3Cc4ccc(C(F)(F)F)cc4C3)cc2=O)CC1]

comprising the steps of
   i′) preparing a compound of formula (III) according to any of the processes as defined above;
   ii′) reacting a compound of formula (III)

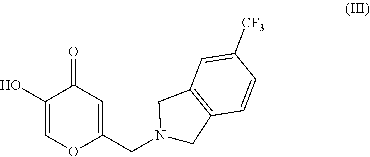 [SMILES: O=c1cc(CN2Cc3ccc(C(F)(F)F)cc3C2)occ1O]

with a compound of formula (IV)

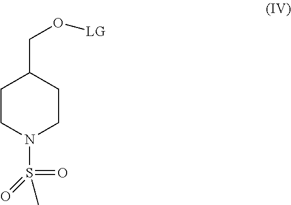

wherein LG is a leaving group selected from a mesyl or a tosyl group,
to produce compound of formula (1A), and optionally converting it to its pharmaceutically acceptable salt.

In still another aspect, the present invention provides a process for the preparation of a compound of formula (1A) or a pharmaceutically acceptable salt thereof

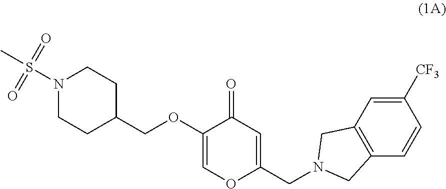 [SMILES: CS(=O)(=O)N1CCC(COc2coc(CN3Cc4ccc(C(F)(F)F)cc4C3)cc2=O)CC1]

comprising the steps of
   i″) reacting a compound of formula (III)

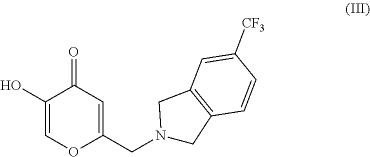 [SMILES: O=c1cc(CN2Cc3ccc(C(F)(F)F)cc3C2)occ1O]

with a compound of formula (IV);

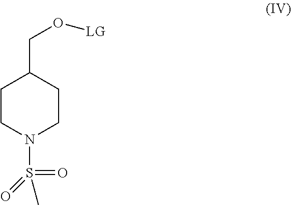

wherein LG is a leaving group selected from a mesyl or a tosyl group,
in dimethyl sulfoxide in the presence of cesium carbonate;
   ii″) adding acetonitrile and water to the mixture; and
   iii″) isolating the compound of formula (1A); and optionally converting it to its pharmaceutically acceptable salt.

In still another aspect, the present invention provides new intermediate compounds of formula (IXa), or of formula (XI) or a pharmaceutically acceptable salt thereof

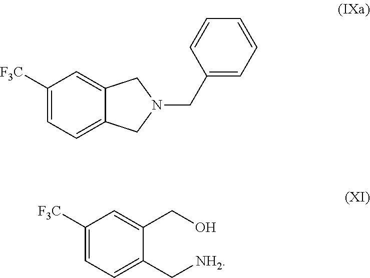 [SMILES: FC(F)(F)c1ccc2c(c1)CN(Cc1ccccc1)C2 ; NCc1ccc(C(F)(F)F)cc1CO]

### DETAILED DESCRIPTION OF THE INVENTION

According to one embodiment of the invention, compound of formula (V) or a pharmaceutically acceptable salt thereof is prepared using a method comprising the steps of
a) treating a compound of formula (VI)

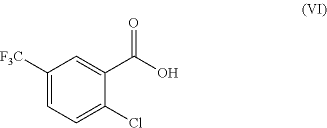 [SMILES: O=C(O)c1cc(C(F)(F)F)ccc1Cl]

with copper cyanide to obtain a compound of formula (VII)

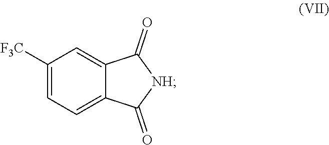 [SMILES: O=C1NC(=O)c2cc(C(F)(F)F)ccc21]

b) protecting the compound of formula (VII) to obtain a compound of formula (VIII) wherein Pg is a protecting group

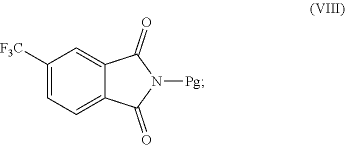

c) reducing the compound of formula (VIII) to obtain a compound of formula (IX)

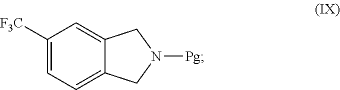

and
d) deprotecting the compound of formula (IX) to obtain a compound of formula (V); and optionally converting it to its pharmaceutically acceptable salt.

For preparing the compound of formula (VII), a compound of formula (VI) and copper cyanide together with a suitable solvent such as dimethylacetamide are charged to the reactor vessel which is preferably under nitrogen atmosphere. If the solvent is dimethylacetamide, the amount of solvent is suitably about 1-3 liter, for example 2 liter, per 1 kg of the starting compound (VI). The amount of copper cyanide is suitably about 0.5-1 kg, for example 0.7 kg, per 1 kg of the starting compound (VI). The reaction is suitably conducted at an elevated temperature ranging typically from about 100 to about 140° C., preferably from about 120 to about 135° C., for example at about 130° C. The mixture is stirred at this temperature for a time period which is generally about 10-24 h, typically about 15-20 h. The mixture can thereafter be cooled, for example, to lower than 45° C. To facilitate the completion of the reaction, a solution of ferric chloride in water and aqueous 30% hydrochloric acid can then be added. The amount of ferric chloride is suitably about 1-2 kg, for example about 1.5 kg, per 1 kg of the starting compound (VI). The mixture may then be heated, for example, to about 65° C. with agitation, for example, for about 1 h. The mixture can then be cooled, for example, to about 30° C. Further water can be added and the mixture is cooled further, for example, to about 5° C., followed by agitation, for example, for about 1 h. The crude precipitated product can be isolated, for example, by filtering and washed with water. The isolated crude product can be transferred to another reactor vessel together with sodium bicarbonate and water, stirred and filtered followed by washing with water and drying the crude product, for example, under reduced pressure at about 50-55° C. The dried crude product can be purified further by mixing it, for example, with ethyl acetate followed by heating the mixture under agitation to about 45° C. and cooling, for example, to about 30° C. The mixture can then be filtered and the solids washed with ethyl acetate. The filtrates can be concentrated, for example, under vacuum at <45° C. The obtained slurry can be cooled, for example, to about 25° C. and methanol and water can be added. The slurry is then suitably stirred and filtered, and the solids washed with water. The obtained solid product of formula (VII) can be dried, for example, under reduced pressure at about 50-55° C.

The compound of formula (VIII) wherein Pg is a protecting group is thereafter prepared. According to one preferred embodiment of the invention, Pg is a benzyl group. Thus, the manufacture of a compound of formula (VIII) wherein Pg is a benzyl group (compound VIIIa) can be carried out by adding to a reactor vessel under nitrogen atmosphere a compound of formula (VII), benzyl bromide and a suitable base such as potassium carbonate together with suitable solvent such as dimethylacetamide. If the solvent is dimethylacetamide, the amount of solvent is suitably about 5 l per 1 kg of the starting compound (VII). The amount of potassium carbonate is suitably about 1-2 kg, for example about 1.6 kg, per 1 kg of starting compound (VII). The reaction is suitably conducted at a temperature of about 20-40° C., for example at about 30° C. The mixture is stirred at this temperature for a time period sufficient to complete the reaction, typically for about 8-15 h. The mixture can thereafter be cooled, for example, to about 20° C. The mixture can then be added to another reactor vessel containing water followed by stirring the mixture for about 3 h and isolating the precipitate, for example by filtering, followed by washing with water and drying, for example at reduced pressure at about 55° C. for about 6 h. To the cooled crude product can be added n-heptane followed by stirring the slurry at about 10° C. The slurry can be filtered and washed with n-heptane and dried under reduced pressure at about 55° C. for about 8 h to obtain the compound of formula (VIIIa).

The reduction of the compound of formula (VIIIa) to a compound of formula (IXa)

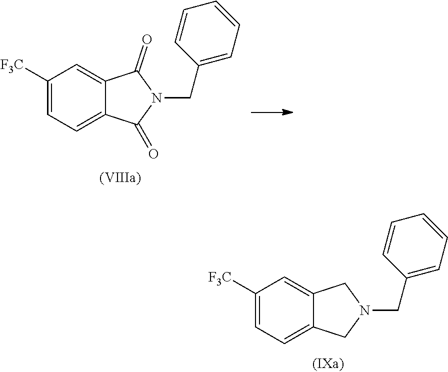 [SMILES: O=C1c2ccc(C(F)(F)F)cc2C(=O)N1Cc1ccccc1 ; FC(F)(F)c1ccc2c(c1)CN(Cc1ccccc1)C2]

is suitably carried out by adding to a reactor vessel under nitrogen atmosphere compound of formula (VIIIa), borohydride salt, for example sodium borohydride, and boron trifluoride tetrahydrofuran complex together with suitable solvent such as tetrahydrofuran. If the solvent is tetrahydrofuran, the amount of solvent is suitably about 25 l per 1 kg of the starting compound (VIIIa). The reaction is suitably conducted at an temperature of about 20-40° C., for example at about 30° C. The amount of sodium borohydride is suitably about 0.5-1.5 kg, for example about 1 kg, per 1 kg of starting compound (VIIIa). The amount of boron trifluoride tetrahydrofuran complex is suitably about 3-5 kg, for example about 4 kg, per 1 kg of starting compound (VIIIa). The mixture is stirred at this temperature for a time period sufficient to complete the reaction, for example for about 30-50 h. The mixture can then be cooled, for example to about 5° C., and methanol added followed by stirring at about 60° C. for about 2 h. The mixture is then suitably cooled to about 5° C. followed by adding hydrochloric acid and water and concentrating the mixture. To the resulting slurry aqueous sodium hydroxide is added followed by ethyl acetate. The slurry is filtered and the filtrate is suitably charged to a clean reaction vessel. The phases are separated and the organic phase is evaporated to dryness. To the crude product n-heptane and activated charcoal is added followed by stirring the mixture at about 30° C. for about 1 h. The solids are filtered and the filtrate can be evaporated to dryness to obtain the compound of formula (IXa).

Deprotection of the compound of formula (IXa) to obtain the compound of formula (V) can be carried out, for example, by hydrogenation the mixture containing the compound of formula (IXa), a suitable catalyst, for example a palladium catalyst such as 10% Pd in carbon, in a suitable solvent such as methanol, using the hydrogenation pressure of, for example, 8-10 bar and the temperature of about 40° C. The amount of methanol is suitably about 8-12 liter, for example 10 liter, per 1 kg of the starting compound (IXa). The hydrogenation is continued until the completion of the reaction, for example about 8 h. The mixture is then cooled, for example to about 30° C. and filtered. The wet catalyst cake can be washed with methanol and the filtrate evaporated to dryness under reduced pressure at <40° C. Ethanol can be added to the residue followed by evaporation to dryness to obtain the compound of formula (V).

The compound of formula (V) can be converted it to its pharmaceutically acceptable salt, for example hydrochloride salt, by adding suitable solvent, for example methyl tert-butyl ether, to the compound of formula (V) followed by 25% ethanolic hydrogen chloride. The mixture is heated to about 40° C. under stirring followed by cooling, for example to about 10° C. The mixture can be concentrated into a thick slurry under reduced pressure at <40° C. and filtered, washed and dried for example at about 40° C. for about 6 h to obtain the hydrochloride salt of the compound of formula (V).

The above synthesis route for preparing the compound of formula (V) or a pharmaceutically acceptable salt thereof has the advantage of using commercially available and easily processable starting materials. The synthesis route is also practical, economical and suitable for use in the large scale and affords the end product in acceptable yield and high purity.

Alternatively, the compound of formula (V) or a pharmaceutically acceptable salt thereof, can be prepared by a method comprising the steps of
a′) treating a compound of formula (VI)

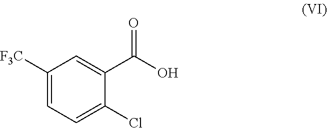 [SMILES: O=C(O)c1cc(C(F)(F)F)ccc1Cl]

with hexacyanoferrate[II] salt in the presence of a catalyst to obtain a compound of formula (X) or a pharmaceutically acceptable salt thereof

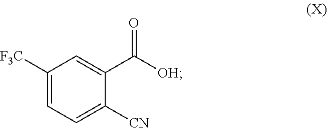 [SMILES: N#Cc1ccc(C(F)(F)F)cc1C(=O)O]

b′) reducing the compound of formula (X) or a pharmaceutically acceptable salt thereof to obtain a compound of formula (XI) or a pharmaceutically acceptable salt thereof

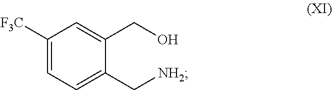 [SMILES: NCc1ccc(C(F)(F)F)cc1CO]

c′) treating the compound of formula (XI) or a pharmaceutically acceptable salt thereof with thionyl chloride to obtain a compound of formula (V); and optionally converting it to its pharmaceutically acceptable salt.

For preparing the compound of formula (X), a suitable solvent such as a mixture of dimethylacetamide, xylene and water, and a suitable base, for example N,N-diisopropylethylamine (DIPEA), are first added to a reactor vessel which is preferably under nitrogen atmosphere. The solvents are then preferably degassed with several vacuum-nitrogen cycles. Thereafter, a suitable catalyst such as palladium catalyst, for example palladium(II) acetate, and a suitable dialkylbiaryl phosphine ligand, for example ditert-butyl-(2-phenylphenyl)phosphane (JohnPhos-ligand) are added. After stirring, the compound of formula (VI) and a hexacyanoferrate[II] salt, for example potassium hexacyanoferrate(II) trihydrate, are added and the mixture heated to about 90-100° C. and stirred until completion of the reaction, typically for a period of about 2-4 h. The mixture is then cooled and filtered, the solids washed with xylene and the filtrate collected.

According to one embodiment, the compound of formula (X) can be isolated from the filtrate, for example, by adding to the filtrate dichloromethane (DCM) and celite followed by filtration. The pH of the filtrate is then adjusted to <1 by aqueous 30% HCl followed by separating the phases. Water can then be added to the organic phase followed by again adjusting the pH to <1 by aqueous 30% HCl and separating the phases. Water is then added to the organic phase followed by adjusting the pH to >12 by aqueous 50% NaOH and separating the phases. The pH of the aqueous phase is then adjusted to <1 by aqueous 30% HCl. Finally, dichloromethane is added and the phases separated. The organic phase is evaporated to dryness to obtain compound of formula (X).

According to another embodiment, the compound of formula (X) can be isolated from the filtrate as its salt, for example sodium salt, by adjusting the pH of the filtrate to about 10-12 with a suitable base, for example aqueous 50% NaOH solution. The formed solids can be filtered, washed with xylene and dried, for example, under reduced pressure at about 40° C. to obtain the compound of formula (X) as a sodium salt. If desired, the compound of formula (X) can be liberated from its salt, for example sodium salt, by dissolving the salt to a suitable solvent, for example dichloromethane (DCM) or ethyl acetate. Water is then added followed by adjusting the pH to <3 by aqueous HCl and separating the phases. The compound of formula (X) can then be isolated from the organic phase, for example by evaporating the organic phase to dryness.

The reduction of the compound of formula (X) or a salt thereof, for example a sodium salt, is suitably carried out by adding to a reactor vessel under nitrogen atmosphere compound of formula (X) and suitable solvent such as tetrahydrofuran (THF). The mixture is suitably cooled, for example to about 10° C. Then a borohydride salt, for example lithium borohydride solution in tetrahydrofuran, is added at <15° C. followed by addition of boron trifluoride tetrahydrofuran (BF₃-THF) complex at <20° C. The mixture can be heated to about 50-70° C. and stirred until completion of the reaction, typically for about 3-5 h. The mixture can then be cooled, for example to about 20° C., followed by addition of methanol and water at <40° C. The pH of the reaction mixture is suitably shifted to about pH 11, for example by addition of aqueous NaOH solution. The solution can be concentrated under vacuum followed by addition of dichloromethane (DCM) and water. The phases are then separated and ethyl acetate and sodium sulphate are added to the aqueous phase. The phases are separated and the combined organic phases can be evaporated to dryness under vacuum to obtain the compound of formula (XI).

The compound of formula (XI) can also be isolated as its salt, for example a hydrochloride salt. In that case the compound of formula (X) or salt thereof is reducted as described above, followed by addition of HCl in ethanol to the organic phase which contains compound of formula (XI). Subsequent evaporation of the organic phase yields the compound of formula (XI) as its hydrochloride salt.

Alternative, the compound of formula (XI) or a salt thereof can be prepared from the compound of formula (X) or a salt thereof, by forming a 2-carbamoyl-5-(trifluoromethyl)benzoic acid (XI′) as a reduction intermediate.

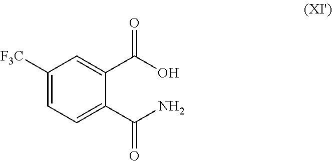 [SMILES: NC(=O)c1ccc(C(F)(F)F)cc1C(=O)O]

The compound of formula (XI′) can be formed by dissolving the compound of formula (X) or a salt thereof, for example a sodium salt, in a suitable solvent, for example tert-butanol, in the presence of a suitable base, for example potassium hydroxide or sodium hydroxide. The mixture can be heated to about 80-90° C. and stirred until completion of the reaction, typically for about 1-3 h. The mixture can then be cooled and aqueous HCl added to reach pH 2-3. The mixture can be extracted, for example with EtOAc, and the organic phases can be evaporated to obtain the compound of formula (XI′). The reduction of the compound of formula (XI′) to compound of formula (XI) is suitably carried out by treating the compound of formula (XI′) with a borohydride salt, for example lithium borohydride, and a BF₃-THF-complex, in a suitable solvent such as tetrahydrofuran (THF). The mixture can be warmed to about 50-70° C. and stirred until completion of the reaction, typically for about 3-5 h. The mixture can then be cooled, for example to about 0° C., followed by addition of methanol and water. The pH of the reaction mixture is suitably shifted to about pH 10, for example by addition of aqueous NaOH solution. The solution can be concentrated under vacuum followed by addition of dichloromethane (DCM) and water. The phases can be separated and the water phase extracted with DCM. The combined organic phases can be evaporated to dryness under vacuum to obtain the compound of formula (XI). If desired, the compound of formula (XI) can be isolated as hydrochloride salt by addition of HCl-EtOH to the organic phase before evaporation of the organic phase to dryness.

For obtaining the compound of formula (V), the compound of formula (XI) or a salt thereof and a suitable solvent, for example, isopropyl acetate is added to the reaction vessel. Thionyl chloride in isopropyl acetate is added slowly to the mixture, for example within 1 h, at about 20° C. The reaction mixture is stirred until completion, typically for about 1-4 h. Thereafter, NaOH solution is added slowly, for example within 1 h, at about 20° C. The formed phases are separated and the organic phase is evaporated to dryness, for example under reduced pressure to obtain the compound of formula (V).

The compound of formula (V) can be converted it to its pharmaceutically acceptable salt, for example hydrochloride salt, by adding suitable solvent, for example ethyl acetate, to the compound of formula (V) followed by slowly adding ethanolic hydrogen chloride until precipitation follows. The mixture is filtered, washed with hexane and ethyl acetate and dried for example under vacuum at 40° C. to obtain the hydrochloride salt of the compound of formula (V).

The above synthesis route for preparing the compound of formula (V) or a pharmaceutically acceptable salt thereof has the advantage of using commercially available and easily processable starting materials and having low number of synthesis steps affording the end product in high purity.

According to one embodiment of the invention a compound of formula (III)

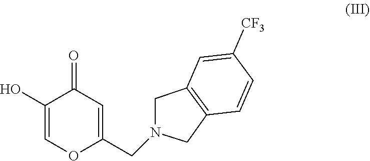 [SMILES: O=c1cc(CN2Cc3ccc(C(F)(F)F)cc3C2)occ1O]

is prepared using a method comprising the steps of
   a″) reacting a compound of formula (XII)

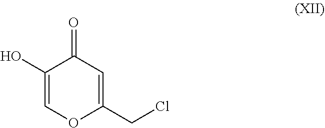

with hydrochloride salt of 5-(trifluoromethyl)isoindoline (V) in dimethyl sulfoxide in the presence of N,N-diisopropylethylamine;
   b″) adding isopropyl alcohol and water to the mixture; and
   c″) isolating the compound of formula (III).

This method can be carried out by adding a compound of formula (XII), hydrochloride salt of 5-(trifluoromethyl)isoindoline (V), N,N-diisopropylethylamine (DIPEA) and dimethyl sulfoxide to the reactor vessel under nitrogen atmosphere. The amount of dimethyl sulfoxide is suitably about 350-450 ml, for example 400 ml, per 100 g of the starting compound (XII). The amount of DIPEA is suitably about 200-400 ml, for example 350 ml, per 100 g of the starting compound (XII). The mixture is then stirred at a temperature which is typically from about 30° C. to about 50° C., for example 40±5° C., for a time period sufficient to complete the reaction. The reaction time ranges generally from about 1 h to about 6 h, typically about 2-4 h. Thereafter a mixture of isopropyl alcohol and water optionally with acetic acid are added to the reaction mixture. The ratio of isopropyl alcohol to water is suitably from about 1:2 to about 1:4, for example about 1:3, per volume. The mixture can be seeded, if desired. The mixture is then stirred for about 1-2 h at 40±5° C. and then cooled to a temperature which is from about 0° C. to about 20° C., for example 10±5° C. The cooling is suitably carried out during about 2 to 4 h, for example during about 2.5 h. The mixture is stirred for a period sufficient to complete the precipitation, for example about 1 h. The precipitated mass can be isolated, for example by filtering, washed with water and cold isopropanol. The product can be dried under reduced pressure at about 40-60° C. to obtain the compound of formula (III).

The above method for preparing the compound of formula (III) has the advantage of simple work-up and excellent yield.

Compound of formula (XII) can suitably be prepared by reacting a compound of formula (XIII)

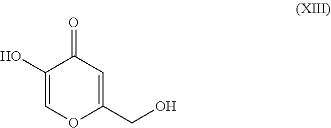

with thionyl chloride in acetonitrile, adding water, cooling the mixture and isolating the compound of formula (XIII).

For carrying out this method acetonitrile and kojic acid (XIII) are added to the reaction vessel under nitrogen. The amount of acetonitrile is suitably about 350 ml per 100 g of the starting compound (XIII). The mixture is suitably heated to a temperature which is from about 30° C. to about 60° C., for example 45±5° C. Thionyl chloride is then added slowly, for example during about 0.5-1 h, while keeping temperature at about 45±5° C. The mixture is stirred for a time period sufficient to complete the reaction, for example about 0.5-1 h. Water is then added slowly, for example over about 0.5-1 h, while keeping temperature at about 45±5° C. The ratio of water to acetonitrile is suitably from about 1:1.5 to about 1:2, for example about 1:1.75. The mixture is stirred at this temperature for least 0.5 h before cooling, for example to a temperature which is from about 0° C. to about 10° C. The cooling is carried out slowly, for example during 2 to 8 h. The precipitated mass can then be isolated, for example by filtering, washed with water and acetonitrile and dried, for example under reduced pressure at about 40-60° C., to afford compound of formula (XII).

The compound of formula (1A) or a pharmaceutically acceptable salt thereof

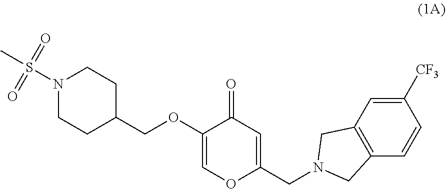 [SMILES: CS(=O)(=O)N1CCC(COc2coc(CN3Cc4ccc(C(F)(F)F)cc4C3)cc2=O)CC1]

can be prepared by reacting the compound of formula (III)

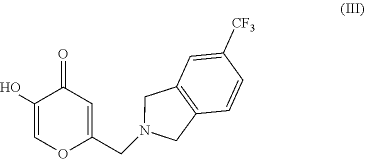 [SMILES: O=c1cc(CN2Cc3ccc(C(F)(F)F)cc3C2)occ1O]

with a compound of formula (IV)

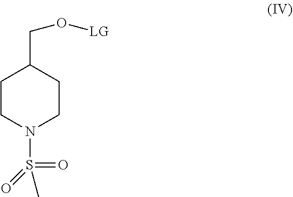

wherein LG is a leaving group selected from a mesyl or a tosyl group, to produce compound of formula (1A), and optionally converting it to its pharmaceutically acceptable salt.

According to one preferred embodiment, the compound of formula (1A) or a pharmaceutically acceptable salt thereof

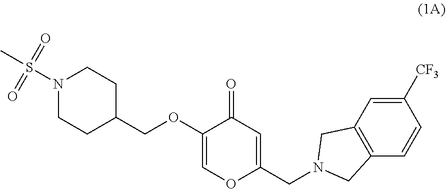 [SMILES: CS(=O)(=O)N1CCC(COc2coc(CN3Cc4ccc(C(F)(F)F)cc4C3)cc2=O)CC1]

is prepared by a method comprising the steps of
   i″) reacting a compound of formula (III)

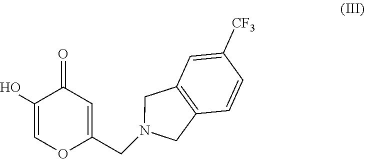 [SMILES: O=c1cc(CN2Cc3ccc(C(F)(F)F)cc3C2)occ1O]

with a compound of formula (IV);

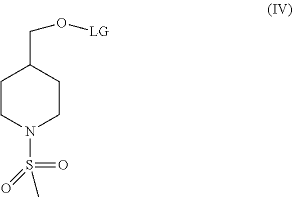

wherein LG is a leaving group selected from a mesyl or a tosyl group,
in dimethyl sulfoxide in the presence of cesium carbonate;
   ii″) adding acetonitrile and water to the mixture; and
   iii″) isolating the compound of formula (1A); and optionally converting it to its pharmaceutically acceptable salt.

This method can be carried out by adding to a reactor under nitrogen atmosphere dimethyl sulfoxide a compound of formula (III), a compound of formula (IV) and cesium carbonate. The amount of dimethyl sulfoxide is suitably 5 l per 1 kg of starting compound (III). The reaction is preferably conducted at an elevated temperature, for example at the temperature from about 40° C. to about 80° C., for example at about 50-75° C. The reaction mixture is mixed at this temperature until completion of the reaction. The reaction time ranges generally from about 1 h to about 6 h, typically from about 2 h to about 3 h. Acetonitrile and water are then added at about 40° C. to about 80° C., for example at about 50-75° C. The mixture is cooled and, if desired, seeded, for example, at about 50 to 70° C. Further water can be added and the suspension is stirred for about 1-2 h, for example, at about 50 to 70° C. The ratio of acetonitrile to water at the end of water addition is suitably in the range of about 1:1.5 to 1:2, per volume. Thereafter, the mixture is slowly cooled to a temperature which may typically range from about 5° C. to about 25° C., for example 20±5° C. The cooling is suitably carried out during about 2 to 4 h, for example during about 3.0±0.5 h. The mixture is then stirred for a period sufficient to complete precipitation, for example about 2-8 h, prior to isolation of the end product, for example by filtering. The product can be washed with water and cold isopropanol. The isolated wet solids can be dried, for example, under reduced pressure at about 40-60° C. to afford the compound of formula (1A).

If desired, compound (1A) may be converted to a pharmaceutically acceptable salt thereof by methods known in the art.

The above method for preparing the compound for formula (1A) has the advantage of providing the possibility to crystallize the end product directly from the reaction solvent with good yield, low color and high purity without the need to purify the end product by column chromatography.

Compound of formula (IV′)

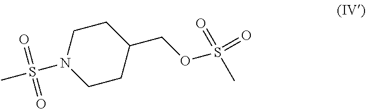 [SMILES: CS(=O)(=O)OCC1CCN(S(C)(=O)=O)CC1]

can be prepared by a method comprising the steps of
   a) reacting a compound of formula (IVa)

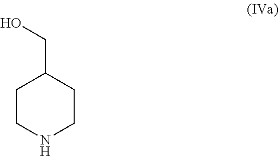

with methanesulfonyl chloride in acetonitrile-pyridine solvent;
   b) adding water and acetic acid to the mixture; and
   c) isolating the compound of formula (IV′).

The method can be carried out by adding to a reactor vessel under nitrogen acetonitrile, pyridine and piperidin-4-ylmethanol (IVa). The ratio of acetonitrile to pyridine at step a) is typically from about 1:2 to about 2:1, for example about 1:1, per volume. The amount of acetonitrile/pyridine mixture is suitably from about 600 ml to about 700 ml per 100 g of the starting compound (IVa). Methanesulfonyl chloride is added slowly, for example during 0.5-1 h, while keeping temperature below 35° C. The temperature of the mixture may then be adjusted to about 25-50° C., for example to 35±5° C., and stirred for a time period sufficient to complete the reaction. The reaction time is generally from about 1 h to about 6 h, typically about 2-3 h. Thereafter water is rapidly added to the mixture followed by acetic acid. The ratio of water to acetic acid may be from about 5:1 to about 10:1, for example about 7:1, per volume. The mixture is then cooled to a temperature which is from about −10° C. to about 10° C., for example 0±5° C. The cooling may be carried out during about 1 to 6 h, for example during about 3 h followed by stirring for a period sufficient to complete precipitation, for example about 1 h, prior to isolation of the end product, for example by filtering. The precipitated product can be washed with water and dried, for example under reduced pressure at about 40-60° C., to afford crystalline compound of formula (IV).

Compound of formula (IV″)

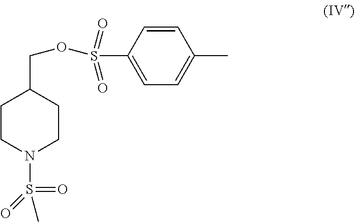 [SMILES: Cc1ccc(S(=O)(=O)OCC2CCN(S(C)(=O)=O)CC2)cc1]

can be prepared by a method comprising the steps of
   a) reacting a compound of formula (IVa)

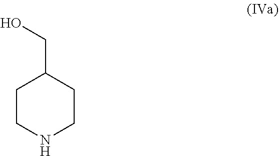

with chlorotrimethylsilane to obtain compound of formula (IVb)

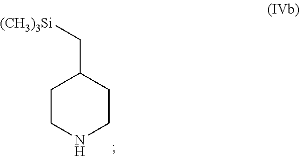

b) reacting the compound of formula (IVb) with methanesulfonyl chloride to obtain a compound of formula (IVc)

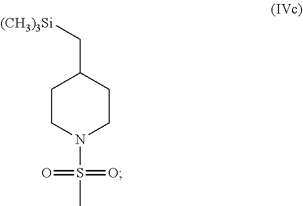

c) treating the compound of formula (IVc) with p-toluenesulfonic acid in the presence of methanol to obtain a compound of formula (IVd)

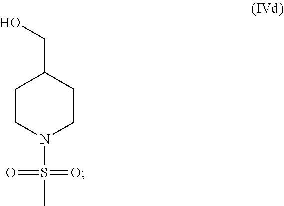

and
d) reacting the compound of formula (IVd) with p-toluenesulfonyl chloride to obtain the compound of formula (IV″).

The method can be carried out by adding to a reactor vessel under nitrogen atmosphere piperidin-4-ylmethanol, suitable solvent such as dichloromethane and a base such as 1,1,3,3-tetramethylguanidine. Chlorotrimethylsilane is then added gradually, for example during 1 h while keeping the temperature under 25° C. After stirring, for example for about 1 h, a base such as N-methylmorpholine is added followed by cooling the mixture, for example, to below 10° C. Methanesulfonyl chloride is then added slowly, for example during about 2 h, while keeping the temperature under 25° C. After the reaction is complete, the reaction can be quenched, for example, by adding 5% aqueous ammonia. The organic layer is isolated and combined with water followed by adjusting pH to 5-6, for example with citric acid. The organic layer is recovered and p-toluenesulfonic acid, for example in the form of monohydrate, is added together with methanol. Part of the solvent may be distilled off, acetonitrile is suitably added followed by further distillation of the solvent. The residue is allowed to cool and pyridine is added. The mixture is then added slowly, for example during about 1.5 h, to a mixture of pyridine and p-toluenesulfonyl chloride while keeping the temperature below 40° C. followed by stirring. Water is then added and the slurry is cooled, for example to about 0° C., during several hours, for example during about 3 h. The mixture can then be stirred for a period sufficient to complete precipitation, for example about 2 h, prior to isolation of the end product, for example by filtering. The precipitate can be washed with water and ice-cold isopropanol and dried, for example under reduced pressure at about 40-50° C., to afford compound of formula (IV″).

The invention is further illustrated by the following non-limiting examples.

### Example 1. Preparation of 2-(Chloromethyl)-5-hydroxy-4H-pyran-4-one (XII)

To a reactor under nitrogen was added acetonitrile (525 ml) and kojic acid (XIII) (150 g). The mixture was heated to 45±5° C. Thionyl chloride (85 ml) was added over about 30 min while keeping temperature at 45±5° C. The mixture was agitated until completion of the reaction, for about 30 min. Water (300 ml) was added slowly over about 30 min while keeping temperature at 45±5° C. followed by mixing for about 1 h. The suspension was cooled to 5±5° C. over several hours followed by mixing for about 1 h. The product was collected and washed with water (450 ml) and acetonitrile (375 ml). The product was dried at 40-60° C. under vacuum to afford 143.7 g (84.8%) of the title compound.

### Example 2. Preparation of 5-(Trifluoromethyl)isoindoline-1,3-dione (VII)

To a reactor under nitrogen was charged dimethylacetamide (425 l), 2-chloro-5-(trifluoromethyl)benzoic acid (212 kg) and copper cyanide (146 kg). The reaction mixture was heated to 128° C. and agitated for about 17 h. The mixture was cooled to <45° C. and a solution of ferric chloride (308 kg) in water (531 l) was added at <50° C., followed by an addition of aqueous 30% hydrochloric acid (122 kg) at <50° C. The mixture was heated to 65° C. and agitated for about 1 h. The mixture was cooled to 30° C. and water (2124 l) was added. The mixture was further cooled to 5° C., agitated for about 1 h and the crude product was filtered and washed with water (212 l). To a reactor under nitrogen was charged sodium bicarbonate (53 kg), water (1062 l) and the isolated crude product. The mixture was stirred for about 0.5 h, filtered, washed with water (212 l) and dried under vacuum at 50-55° C. The dried crude product and ethyl acetate (2124 l) was charged to a reactor and the mixture was heated to 45° C. and agitated for about 0.5 h. The mixture was cooled to 30° C., filtered and the solids were washed with ethyl acetate (425 l). The filtrates were concentrated under vacuum at <45° C. After the distillation the slurry was cooled to 25° C. and methanol (956 l) and water (956 l) were added. The slurry was stirred for about 1 h at 25° C. and filtered. The solids were washed with water (106 l) and dried under vacuum at 50-55° C. Yield 156 kg (74%) of the title compound.

### Example 3. Preparation of 2-Benzyl-5-(trifluoromethyl)isoindoline-1,3-dione (VIIIa)

To a reactor under nitrogen was charged dimethylacetamide (250 l), 5-(tri-fluoromethyl)isoindoline-1,3-dione (49.9 kg), potassium carbonate (80 kg) and benzyl bromide (60 kg). The reaction mixture was heated to 30° C., agitated until completion, for about 11 h, and cooled to 20° C. The reaction mixture was added to another reactor containing water (749 l) at 25° C. The mixture was stirred for about 3 h at 25° C., filtered with a filter dryer, washed with water (50 l), dried for about 6 h at 55° C. and cooled to 10° C. To the filter dryer was added n-heptane (200 l) and the slurry was stirred for about 1 h at 10° C. The slurry was filtered and washed with n-heptane (50 l) and dried under vacuum at 55° C. for about 8 h. Yield 58 kg (83%) of the title compound.

### Example 4. Preparation of 2-Benzyl-5-(trifluoromethyl)isoindoline (IXa)

To a reactor flushed with nitrogen was charged tetrahydrofuran (5000 l), 2-benzyl-5-(trifluoromethyl)isoindoline-1,3-dione (200 kg), sodium borohydride (206 kg) and boron trifluoride tetrahydrofuran complex (828 kg) at 30° C. The reaction mixture was stirred until completion, for about 40 h. The mixture was cooled to 5° C. and methanol (1000 l) was added. The mixture was heated to 60° C., stirred for about 2 h and cooled to 5° C. Hydrochloric acid (530 l) and water (1100 l) were added and the mixture was concentrated to a thick slurry. To the slurry was added sodium hydroxide (400 kg) dissolved in water (1500 l), followed by ethyl acetate (1000 l). The slurry was filtered and the solids washed twice with ethyl acetate (2×400 l). The filtrates were charged to a clean reactor, the phases were separated and the aqueous layer was backextracted with the ethyl acetate washings. All organic phases were combined and evaporated to dryness. To the crude material was added n-heptane (200 l) and the mixture was evaporated to dryness. To the crude material was added n-heptane (200 l) and activated charcoal (20 kg) and the mixture was stirred at 30° C. for about 1 h. The solids were filtered and the cake was washed with n-heptane (200 l). The filtrate and the washings were combined and evaporated to dryness. Yield 134 kg (67%) of the title compound.

### Example 5. Preparation of Hydrochloride Salt of 5-(trifluoromethyl)isoindoline (V)

To a reactor under nitrogen was added methanol (1090 l), 2-benzyl-5-(tri-fluoromethyl)isoindoline (109 kg) and 10% Pd/C (11 kg). The reaction mixture was hydrogenated at 40° C. and 8-10 bar until completion, for about 8 h. The mixture was cooled to 30° C. and filtered. The wet catalyst cake was washed with methanol (218 l) and the filtrate was evaporated to dryness under vacuum at <40° C. To the residue was added ethanol (218 l) and the mixture was evaporated to dryness under vacuum at <40° C. To the residue was added methyl tert-butyl ether (763 l). The mixture was cooled to 10° C. and 25% ethanolic hydrogen chloride (109 l) was added. The mixture was heated to 40° C., stirred for about 15 min, cooled to 10° C. and stirred for about 1.5 h. The mixture was concentrated into a thick slurry under vacuum at <40° C. and filtered. The wet cake was washed with methyl tert-butyl ether (109 l) and dried at 40° C. for about 6 h. Yield 36.5 kg (34%) of the title compound.

### Example 6. Preparation of Sodium Salt of 2-Cyano-5-(trifluoromethyl)benzoic acid (X)

To a reactor under nitrogen was charged dimethylacetamide (20 ml), xylene (40 ml), water (3.8 ml) and DIPEA (6.3 ml). The solvents were degassed with three vacuum-nitrogen cycles. To the degassed solvents was added palladium(II) acetate (0.34 g) and JohnPhos-ligand (0.98 g). The mixture was stirred for about 0.5 h, after which 2-chloro-5-(trifluoromethyl)benzoic acid (5 g) and potassium hexacyano-ferrate(II) trihydrate (7.26 g) were added. The mixture was heated to 95° C. and stirred until completion, for about 3 h. The mixture was cooled to 20° C., filtered and the solids were washed with xylene (15 ml). The pH of the filtrate was adjusted to 10-12 with 50% NaOH-solution (3 ml). The formed solids were filtered, washed with xylene (10 ml) and dried under vacuum at 40° C. Yield 2.3 g (46.5%) of the title compound.

### Example 7. Preparation of 2-Cyano-5-(trifluoromethyl)benzoic acid (X)

To a reaction vessel was added sodium salt of 2-cyano-5-(trifluoromethyl)-benzoic acid (0.20 g), water (3 ml) and dichloromethane (5 ml) followed by stirring the mixture at RT. Then 3% aqueous HCl was added until the pH of the water phase was 2-3. The phases were separated and the water phase extracted with dichloromethane (2×5 ml). The organic phases were combined and evaporated to give the title compound as a white solid (0.14 g, 79%).

### Example 8. Preparation of (2-(Aminomethyl)-5-(trifluoromethyl)phenyl)-methanol (XI)

To a reactor under nitrogen was charged tetrahydrofuran (7 ml) and 2-cyano-5-(trifluoromethyl)benzoic acid (1 g). The mixture was cooled to 10° C. and 2 M lithium borohydride solution in tetrahydrofuran (10.2 ml) was added at <15° C., followed by addition of BF₃-THF-complex (15.3 ml) at <20° C. The reaction mixture was heated to 60° C. and agitated until completion, about 4 h. The mixture was then cooled to 20° C. and methanol (5 ml) and water (8 ml) were added at <40° C. The solution was concentrated under vacuum and dichloromethane (15 ml) and water (15 ml) were added. The phases were separated and ethyl acetate (15 ml) and sodium sulphate (20 g) were added to the aqueous phase. The phases were separated and the organic phases were combined and evaporated to dryness under vacuum. Yield 0.7 g (76%) of the title compound.

### Example 9. Preparation of (2-(Aminomethyl)-5-(trifluoromethyl)phenyl)-methanol (XI) (Alternative Method)

To a reaction vessel under nitrogen was added sodium salt of 2-cyano-5-(tri-fluoromethyl)benzoic acid (0.50 g) and tetrahydrofuran (9.0 ml) at 0° C., after which 2 M lithium borohydride solution in tetrahydrofuran (THF) (4.3 ml) and BF₃-THF-complex (1.4 ml) were added dropwise. The mixture was heated to room temperature (RT) and stirred for 30 min. To drive the reaction to completion, the mixture was heated to 70° C. for about 2 h. The mixture was then cooled to 0° C. followed by a careful addition of methanol (5.0 ml) and water (5.0 ml). The pH of the mixture was shifted to ˜11 by adding a few drops of 50% NaOH. The mixture was then warmed up to 55° C. for 30 min followed by cooling to RT. The mixture was filtered though a filter sheet and rinsed with a small amount of tetrahydrofuran. The volatiles were evaporated and the residue was mixed with dichloromethane (15 ml) and water (10 ml). The phases were separated and the water phase was extracted with dichloromethane (15 ml). The organic phases were combined and evaporated to dryness under vacuum to give the crude title product (0.41 g, 95%).

### Example 10. Preparation of Hydrochloride Salt of (2-(Aminomethyl)-5-(tri-fluoromethyl)phenyl)methanol (XI)

To a reaction vessel under nitrogen was added sodium salt of 2-cyano-5-(tri-fluoromethyl)benzoic acid (0.50 g) and THF (9.0 ml). The slurry was cooled to 0° C., after which 2 M lithium borohydride solution in THF (4.3 ml) and BF₃-THF-complex (1.4 ml) were added dropwise. The mixture was heated to RT and stirred for 30 min. To drive the reaction to completion, the mixture was heated to 70° C. for about 2 h. The mixture was then cooled to 0° C. followed by a careful addition of methanol (5.0 ml) and water (5.0 ml). The pH of the mixture was shifted to ˜11 by adding a few drops of 50% NaOH. The mixture was then warmed to RT and the volatiles were evaporated. The residue was mixed with DCM (15 ml) and water (10 ml). The phases were separated and the water phase was extracted with DCM (15 ml). The organic phases were combined and cooled to 0° C. followed by addition of HCl-EtOH (2.0 ml). The volatiles were evaporated to give the crude title compound (0.43 g, 84%).

### Example 11. Preparation of Hydrochloride Salt of (2-(Aminomethyl)-5-(tri-fluoromethyl)phenyl)methanol (XI) (Alternative Method)

To a reaction vessel under nitrogen was added crude 2-cyano-5-(trifluoro-methyl)benzoic acid (0.14 g) and THF (3.0 ml). The mixture was cooled to 0° C., after which 2 M lithium borohydride solution in THF (1.3 ml) and BF₃-THF-complex (0.43 ml) were added dropwise. The mixture was heated to RT and stirred for 1 h. To drive the reaction to completion, the mixture was heated to 70° C. for about 1.5 h. The mixture was then cooled to RT for 1 hour and then to 0° C., followed by a careful addition of methanol (2.0 ml) and water (2.0 ml). The pH of the mixture was shifted to ˜11 by adding a few drops of 50% NaOH. The mixture was then warmed to RT and the volatiles were evaporated. The residue was mixed with DCM (10 ml) and water (5 ml). The phases were separated and the water phase was extracted with DCM (10 ml). The organic phases were combined and cooled to 0° C. followed by addition of HCl-EtOH (1.0 ml). The volatiles were evaporated to give the title compound (0.17 g, 109%).

### Example 12. Preparation of Hydrochloride Salt of (2-(Aminomethyl)-5-(tri-fluoromethyl)phenyl)methanol (XI) (Alternative Method)

To a reaction vessel under nitrogen was added sodium salt of 2-cyano-5-(tri-fluoromethyl)benzoic acid (0.50 g), potassium hydroxide (0.60 g) and tert-butanol (3.5 ml). The mixture was heated to 85° C. and stirred until the full consumption of the starting material, for about 2 h. The mixture was then cooled to RT, and 3% aqueous HCl was added until the pH was 2-3. The mixture was extracted with EtOAc (3×5 ml). The organic phases were combined and evaporated to give crude 2-carbamoyl-5-(trifluoromethyl)benzoic acid (XI′) (0.45 g, 91%). The product of the previous step and tetrahydrofuran (8.5 ml) were added to a reaction vessel under nitrogen. The slurry was cooled to 0° C., after which 2 M lithium borohydride solution in THF (4.0 ml) and BF₃-THF-complex (1.4 ml) were added dropwise. The mixture was heated to RT and stirred for 1.5 h. To drive the reaction to completion, the mixture is heated to 68° C. for about 3 h. The mixture was then cooled to 0° C., followed by a careful addition of methanol (5.0 ml) and water (5.0 ml). The pH of the mixture was shifted to ˜10 by adding a few drops of 50% aqueous NaOH, after which the mixture was warmed to 55° C. for 20 min and finally cooled to RT. The mixture was filtered through a filter sheet and rinsed with a small amount of THF. The volatiles were evaporated and the residue mixed with DCM (15 ml) and water (5 ml). The phases were separated and the water phase was extracted with DCM (15 ml). The organic phases were combined and cooled to 0° C. followed by addition of HCl-EtOH (1.8 ml). The volatiles were evaporated to give the crude title compound (0.32 g, 69%).

### Example 13. Preparation of Hydrochloride Salt of 5-(trifluoromethyl)-isoindoline (V) (Alternative Method)

To a reaction vessel was added (2-(aminomethyl)-5-(trifluoromethyl)phenyl)-methanol (0.2 g) and isopropyl acetate (1 ml), after which a mixture of isopropyl acetate (1 ml) and thionyl chloride (0.09 ml) was added to the reaction mixture at 20° C. over about 1 h. After the addition, the reaction mixture was stirred until completion, for about 2 h. 50% NaOH-solution (0.4 ml) was added at 20° C. over about 1 h. The formed phases were separated and the organic phase was evaporated to dryness under vacuum. The crude product was dissolved in ethyl acetate (1 ml) and hydrochloric acid-ethanol solution was added dropwise until solids started to form. The product was filtered, washed three times with hexane (3×2 ml) and ethyl acetate (2 ml). Yield 49 mg (23%) of the title compound.

### Example 14. Preparation of Hydrochloride Salt of 5-(trifluoromethyl)isoindoline (V) (Alternative Method)

To a reaction vessel was added thionyl chloride (0.22 ml) and isopropyl acetate (10.0 ml). In another vessel, (2-(aminomethyl)-5-(trifluoromethyl)phenyl)-methanol (0.43 g) was dissolved to isopropyl acetate (10.0 ml), and the solution was added to the reaction vessel within 50 min with a syringe pump at RT. After the addition, the mixture was stirred until the starting material was fully consumed, for about 1.5 h. 5 M aqueous NaOH (2.6 ml) was then added within 45 min at 20° C., after which the mixture was warmed to 30° C. To drive the reaction to completion, 5 M aqueous NaOH (1.3 ml) was added after one hour, within 30 min. The mixture was worked up by adding water (5 ml), separating the phases and washing the organic phase with water (2×5 ml). The organic phase was then cooled to 0° C. and HCl-EtOH (3.0 ml) was added. The volatiles were evaporated and the crude residue was mixed with isopropanol (2.0 ml) with sonication. The mixture was cooled to 0° C. for 10 min and filtered with a sintered funnel. The residue was washed with cold isopropanol (2×0.5 ml) and dried under vacuum to give the crude title compound (97 mg, 20%).

### Example 15. Preparation of Hydrochloride Salt of 5-(trifluoromethyl)isoindoline (V) (Alternative Method)

To a reaction vessel was added hydrochloride salt of (2-(aminomethyl)-5-(tri-fluoromethyl)phenyl)methanol (0.51 g) and isopropyl acetate (10 ml), after which a mixture of isopropyl acetate (11 ml) and thionyl chloride (0.22 ml) was added at RT. After the addition, the mixture was stirred until the starting material was fully consumed, for about 2.5 h. 5 M aqueous NaOH (2.8 ml) was then added within an hour and the mixture was heated to 30° C. To drive the reaction to completion, 5 M aqueous NaOH was added (1.4 ml) after one hour, within 30 min. The mixture was worked up by adding water (5 ml) after which the phases were separated and the organic phase was washed with water (5 ml). The organic phase was then cooled to 0° C. and HCl-EtOH (2.5 ml) was added. The volatiles were evaporated and the crude residue was mixed with isopropanol (2.0 ml) with sonication. The mixture was cooled to 0° C. for 30 min and filtered with a sintered funnel. The residue was washed with cold isopropanol (2×0.5 ml) and dried with suction to give the crude title compound (0.35 g, 73%).

### Example 16. Preparation of 5-hydroxy-2-((5-(trifluoromethyl)isoindolin-2-yl)methyl)-4H-pyran-4-one (III)

To a reactor under nitrogen was added dimethyl sulfoxide (75 ml), 2-(chloro-methyl)-5-hydroxy-4H-pyran-4-one (20 g), 5-(trifluoromethyl)isoindoline HCl (31 g) and N,N-diisopropylethylamine (48 ml). The reaction mixture was heated to 40±5° C. and agitated until completion, for about 3 h. IPA (45 ml), AcOH (3.6 ml) and water (123 ml) were added at 40±5° C. During the water addition the reaction mixture was seeded. The suspension was stirred for about 1 h at 40±5° C., cooled to 10±5° C. over about 2.5 h and mixed for at least 1 h at the end temperature. The product was collected and washed with water (60 ml) and cold isopropanol (60 ml). The isolated wet solids were dried at 40-60° C. under vacuum. Yield 34.3 g (89%) of the title compound.

### Example 17. Preparation of (1-(Methylsulfonyl)piperidin-4-yl)methyl methane sulfonate (IV′)

To a reactor under nitrogen was added acetonitrile (225 ml), pyridine (289 ml) and piperidin-4-ylmethanol (IV) (75 g). The temperature of the mixture was adjusted to 20±5° C. Methanesulfonyl chloride was slowly added over 30 min while keeping temperature below 35° C. The temperature was adjusted to 35±5° C. and the mixture was stirred for 2 h. Water (300 ml) was rapidly added followed by acetic acid (45 ml). The resulting mass was cooled over 3 h to 0±5° C. and mixed for 1 h prior to filtration. The product was washed with water (2 times 225 ml) and dried under vacuum at 40-60° C. to afford 153.3 g (86.8%) of the title compound.

### Example 18. Preparation of (1-(Methylsulfonyl)piperidin-4-yl)methyl 4-methylbenzenesulfonate (IV″)

To a reactor under nitrogen was added dichloromethane (700 ml), piperidin-4-ylmethanol (100 g) and 1,1,3,3-tetramethylguanidine (133 ml). The mixture was agitated until fully dissolved and then cooled below 10° C. Chlorotrimethylsilane (138 ml) was added over about 1 h whilst keeping temperature below 25° C. followed by stirring for about 1 h at 20° C. N-Methylmorpholine (131 ml) was added and the mixture was cooled below 10° C. Methanesulfonyl chloride (82 ml) was added over about 2 h whilst keeping temperature below 25° C. The mixture was then stirred for about 30 min at 20° C. and then quenched with adding 5% aqueous ammonia (500 ml). After brief mixing the layers were separated. The organic layer was combined with water (400 ml) and the pH was adjusted to 5-6 with citric acid (about 35.0 g). The layers were separated and methanol (140 ml) and p-toluenesulfonic acid monohydrate (8.3 g) were added. About 500 ml was distilled off under atmospheric pressure. Acetonitrile (400 ml) was added and the distillation was continued until about 440 ml had been collected (end temperature about 84-85° C.). The residue was allowed to cool to 20° C. and pyridine (100 ml) was added. The solution was transferred to an addition funnel. To another vessel under nitrogen was added pyridine (320 ml) and p-toluenesulfonyl chloride (199 g) and the temperature was adjusted to 35° C. The contents of the addition funnel were added over about 1.5 h whilst keeping temperature below 40° C. followed by stirring for 2 h at 30° C. Water (600 ml) was added slowly. After about 150 ml had been added isopropanol (200 ml) was added to produce more stirrable mixture. The slurry was first heated to 40° C. and then cooled to 0° C. over several hours. The mass was allowed to stir for 2 h prior to filtration. The cake was washed with water (200 ml) and ice-cold isopropanol (200 ml). The product was dried under vacuum at 40-50° C. to give 226.5 g (75.1%) of the title compound.

### Example 19. Preparation of 5-((1-(methylsulfonyl)piperidin-4-yl)methoxy)-2-((5-(trifluoromethyl)isoindolin-2-yl)methyl)-4H-pyran-4-one (1A)

To a reactor under nitrogen was added dimethyl sulfoxide (100 ml), 5-hydroxy-2-((5-(trifluoromethyl)isoindolin-2-yl)methyl)-4H-pyran-4-one (20 g), (1-(methylsulfonyl)piperidin-4-yl)methyl methane sulfonate (20 g) and cesium carbonate (25 g). The reaction mixture was heated to 70±5° C. and agitated until completion, for about 2.5 h. Acetonitrile (60 ml) and water (90 ml) were added at 70±5° C. The mixture was cooled and seeded at 65±3° C. and water (20 ml) was added at the same temperature. The suspension was stirred for 1.5±0.5 h at 65±3° C., cooled to 20±5° C. over 3.0±0.5 h and then mixed for at least 2 h at the end temperature. The product was collected and washed with water (60 ml) and cold isopropanol (40 ml). The isolated wet solids were dried at 40-60° C. under vacuum. Yield 26.7 g (85%) of the title compound.

### Example 20. Preparation of 5-((1-(methylsulfonyl)piperidin-4-yl)methoxy)-2-((5-(trifluoromethyl)isoindolin-2-yl)methyl)-4H-pyran-4-one (1A) (Alternative Method)

To a vessel inerted with nitrogen was charged dimethyl sulfoxide (75 ml), 5-hydroxy-2-((5-(trifluoromethyl)isoindolin-2-yl)methyl)-4H-pyran-4-one (15 g, 48.2 mmol), (1-(methylsulfonyl)piperidin-4-yl)methyl 4-methylbenzenesulfonate (19.26 g, 55.4 mmol) and cesium carbonate (18.84 g, 57.8 mmol). The mixture was heated to 50° C. and agitated until completion, for about 3 h. Acetonitrile (45 ml) was added followed by water (82.5 ml) over about 20 min. The mixture is seeded during the water addition. The resulting slurry was stirred at 50° C. for 1 h after which it was cooled 20° C. over 3 h and stirred overnight. The product was collected by filtration, washed with water (45 ml) and chilled 2-propanol (30 ml). The product was dried under vacuum at 50° C. to give 23.7 g of the title compound.

1. A process for the preparation of a compound of formula (V) or a pharmaceutically acceptable salt thereof 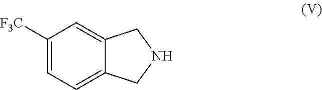 [SMILES: FC(F)(F)c1ccc2c(c1)CNC2]
comprising the steps of either
a) treating a compound of formula (VI) 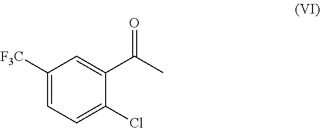
with copper cyanide to obtain a compound of formula (VII) 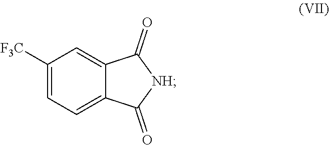 [SMILES: O=C1NC(=O)c2cc(C(F)(F)F)ccc21]
b) protecting the compound of formula (VII) to obtain a compound of formula (VIII) wherein Pg is a protecting group 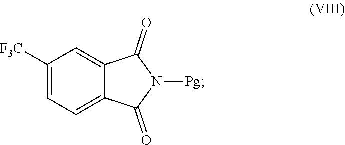
c) reducing the compound of formula (VIII) to obtain a compound of formula (IX) 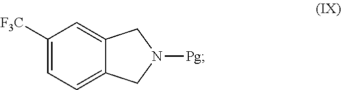
and
d) deprotecting the compound of formula (IX) to obtain a compound of formula (V); and optionally converting it to its pharmaceutically acceptable salt;
or
a′) treating a compound of formula (VI) 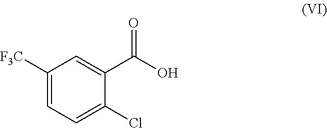 [SMILES: O=C(O)c1cc(C(F)(F)F)ccc1Cl]
with hexacyanoferrate[II] salt in the presence of a catalyst to obtain a compound of formula (X) or a pharmaceutically acceptable salt thereof 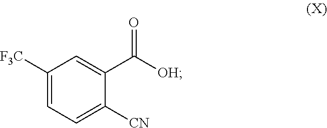 [SMILES: N#Cc1ccc(C(F)(F)F)cc1C(=O)O]
b′) reducing the compound of formula (X) or a pharmaceutically acceptable salt thereof to obtain a compound of formula (XI) or a pharmaceutically acceptable salt thereof 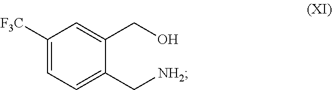 [SMILES: NCc1ccc(C(F)(F)F)cc1CO]
c′) treating the compound of formula (XI) or a pharmaceutically acceptable salt thereof with thionyl chloride to obtain a compound of formula (V); and optionally converting it to its pharmaceutically acceptable salt.

2. The process according to claim 1 comprising the steps of
a) treating a compound of formula (VI) 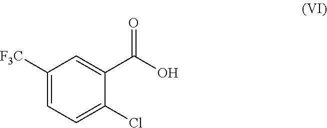 [SMILES: O=C(O)c1cc(C(F)(F)F)ccc1Cl]
with copper cyanide to obtain a compound of formula (VII) 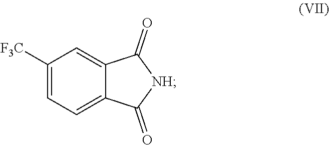 [SMILES: O=C1NC(=O)c2cc(C(F)(F)F)ccc21]
b) protecting the compound of formula (VII) to obtain a compound of formula (VIII) wherein Pg is a protecting group 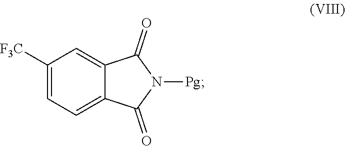
c) reducing the compound of formula (VIII) to obtain a compound of formula (IX) 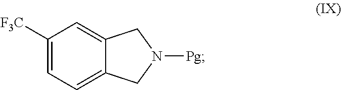
and
d) deprotecting the compound of formula (IX) to obtain a compound of formula (V); and optionally converting it to its pharmaceutically acceptable salt.

3. The process according to claim 1, wherein step a) is carried out in dimethylacetamide.

4. The process according to claim 1, wherein the reaction temperature at step a) is from about 100 to about 140° C., preferably from about 120 to about 135° C.

5. The process according to claim 1, wherein the reaction of step a) is followed by addition of ferric chloride in water and aqueous hydrochloric acid.

6. The process according to claim 1, wherein Pg is a benzyl group.

7. The process according to claim 6, wherein step b) comprises reacting compound of formula (VII) with benzyl bromide in the presence of a base.

8. The process according to claim 7, wherein the base is potassium carbonate.

9. The process according to claim 1, wherein step b) is carried out in dimethylacetamide.

10. The process according to claim 1, wherein step c) comprises treating the compound of formula (VIII) with borohydride salt and boron trifluoride tetrahydrofuran complex.

11. The process according to claim 10, wherein the borohydride salt is sodium borohydride.

12. The process according to claim 1, wherein step c) is carried out in tetrahydrofuran.

13. The process according to claim 1, wherein step d) comprises hydrogenation in the presence of a catalyst.

14. The process according to claim 13, wherein the catalyst is a palladium catalyst.

15. The process according to claim 1, wherein step d) is carried out in methanol.

16. The process according to claim 1, wherein after step d) the obtained compound of formula (V) is converted to its hydrochloride salt.

17. The process according to claim 1 comprising the steps of
a′) treating a compound of formula (VI) 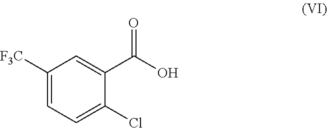 [SMILES: O=C(O)c1cc(C(F)(F)F)ccc1Cl]
with hexacyanoferrate[II] salt in the presence of a catalyst to obtain a compound of formula (X) or a pharmaceutically acceptable salt thereof 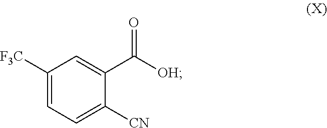 [SMILES: N#Cc1ccc(C(F)(F)F)cc1C(=O)O]
b′) reducing the compound of formula (X) or a pharmaceutically acceptable salt thereof to obtain a compound of formula (XI) or a pharmaceutically acceptable salt thereof 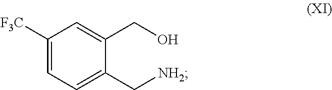 [SMILES: NCc1ccc(C(F)(F)F)cc1CO]
c′) treating the compound of formula (XI) or a pharmaceutically acceptable salt thereof with thionyl chloride to obtain a compound of formula (V); and optionally converting it to its pharmaceutically acceptable salt.

18. The process according to claim 17, wherein the hexacyanoferrate[II] salt in step a′) is potassium hexacyanoferrate[II] trihydrate.

19. The process according to claim 17, wherein the catalyst in step a′) is a palladium catalyst.

20. The process according to claim 17, wherein step a′) is carried out in the presence of a dialkylbiaryl phosphine ligand.

21. The process according to claim 20, wherein the dialkylbiaryl phosphine ligand is ditert-butyl-(2-phenylphenyl)phosphane (JohnPhos-ligand).

22. The process according to claim 17, wherein the base in step a′) is N,N-diisopropylethylamine (DIPEA).

23. The process according to claim 17, wherein step a′) is carried out in the mixture of dimethylacetamide, xylene and water.

24. The process according to claim 17, wherein step b′) comprises treating the compound of formula (X) with borohydride salt and boron trifluoride tetrahydrofuran complex.

25. The process according to claim 24, wherein the borohydride salt is lithium borohydride.

26. The process according to claim 17, wherein step b′) is carried out in tetrahydrofuran.

27. The process according to claim 17, wherein step c′) is carried out in isopropyl acetate.

28. The process according to claim 17, wherein after step c′) the obtained compound of formula (V) is converted to its hydrochloride salt.

29. A process for the preparation of a compound of formula (III) 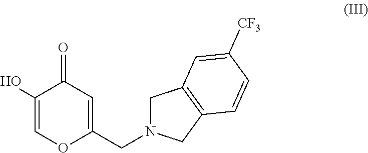 [SMILES: O=c1cc(CN2Cc3ccc(C(F)(F)F)cc3C2)occ1O]
comprising the steps of
a″) reacting a compound of formula (XII) 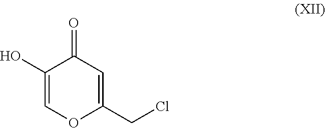
with hydrochloride salt of 5-(trifluoromethyl)isoindoline (V) in dimethyl sulfoxide in the presence of N,N-diisopropylethylamine;
b″) adding isopropyl alcohol and water to the mixture; and
c″) isolating the compound of formula (III).

30. The process according to claim 29, wherein step b″) further comprises addition of acetic acid.

31. The process according to claim 29, wherein before step c″) the mixture is cooled to a temperature which is form about 5° C. to about 15° C.

32. The process according to claim 29, wherein hydrochloride salt of 5-(trifluoromethyl)isoindoline (V) is prepared according to any one of claims 1 to 28.

33. The process according to claim 29, wherein the compound of formula (XII) is prepared by reacting a compound of formula (XIII) 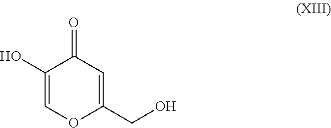
with thionyl chloride in acetonitrile, adding water, and isolating the compound of formula (XII).

34. A process for the preparation of a compound of formula (1A) or a pharmaceutically acceptable salt thereof 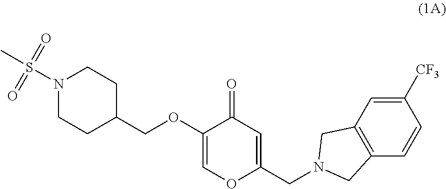 [SMILES: CS(=O)(=O)N1CCC(COc2coc(CN3Cc4ccc(C(F)(F)F)cc4C3)cc2=O)CC1]
comprising the steps of
i) preparing hydrochloride salt of 5-(trifluoromethyl)isoindoline (V) according to the process of claim 1;
ii) reacting hydrochloride salt of 5-(trifluoromethyl)isoindoline (V) with a compound of formula (XII) 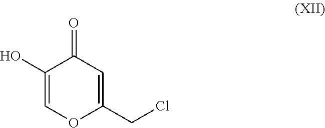
to produce a compound of formula (III); 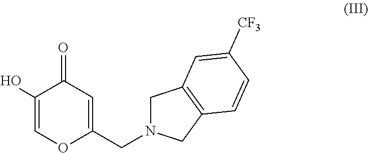 [SMILES: O=c1cc(CN2Cc3ccc(C(F)(F)F)cc3C2)occ1O]
iii) reacting a compound of formula (III) with a compound of formula (IV) 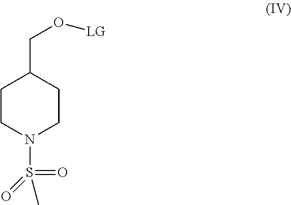
wherein LG is a leaving group selected from a mesyl or a tosyl group,
to produce compound of formula (1A), and optionally converting it to its pharmaceutically acceptable salt.

35. A process for the preparation of a compound of formula (1A) or a pharmaceutically acceptable salt thereof 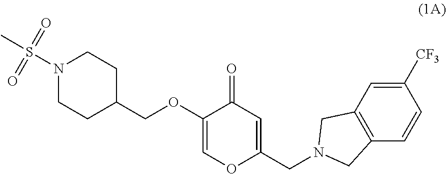 [SMILES: CS(=O)(=O)N1CCC(COc2coc(CN3Cc4ccc(C(F)(F)F)cc4C3)cc2=O)CC1]
comprising the steps of
i′) preparing a compound of formula (III) according to the process of claim 29;
ii′) reacting a compound of formula (III) 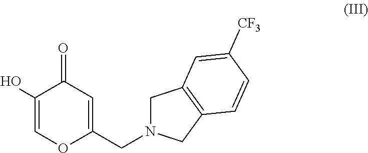 [SMILES: O=c1cc(CN2Cc3ccc(C(F)(F)F)cc3C2)occ1O]
with a compound of formula (IV) 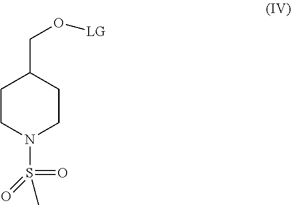
wherein LG is a leaving group selected from a mesyl or a tosyl group,
to produce compound of formula (1A), and optionally converting it to its pharmaceutically acceptable salt.

36. A process for the preparation of a compound of formula (1A) or a pharmaceutically acceptable salt thereof 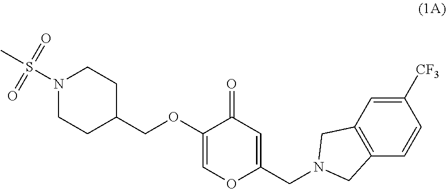 [SMILES: CS(=O)(=O)N1CCC(COc2coc(CN3Cc4ccc(C(F)(F)F)cc4C3)cc2=O)CC1]
comprising the steps of
i″) reacting a compound of formula (III) 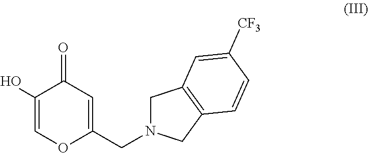 [SMILES: O=c1cc(CN2Cc3ccc(C(F)(F)F)cc3C2)occ1O]
with a compound of formula (IV); 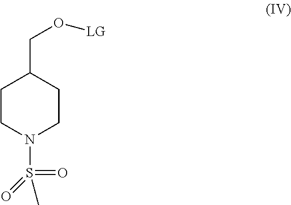
wherein LG is a leaving group selected from a mesyl or a tosyl group,
in dimethyl sulfoxide in the presence of cesium carbonate;
ii″) adding acetonitrile and water to the mixture; and
iii″) isolating the compound of formula (1A); and optionally converting it to its pharmaceutically acceptable salt.

37. A compound of formula (IXa), or (XI) or a pharmaceutically acceptable salt thereof 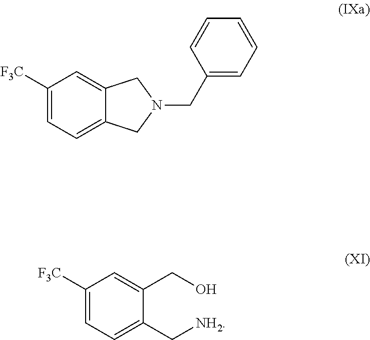 [SMILES: FC(F)(F)c1ccc2c(c1)CN(Cc1ccccc1)C2 ; NCc1ccc(C(F)(F)F)cc1CO]
